(19) United States
(12) Patent Application Publication
(10) Pub. No.: US 2019/0084900 A1
(43) Pub. Date: Mar. 21, 2019

(54) ISOTOPICALLY MODIFIED COMPOSITION AND THERAPEUTIC USES THEREOF

(71) Applicant: RETROTOPE, INC., Los Altos, CA (US)

(72) Inventor: Mikhail Sergeevich Shchepinov, Kingston Upon Thames (GB)

(21) Appl. No.: 16/095,573

(22) PCT Filed: May 1, 2017
(86) PCT No.: PCT/US2017/030446

§ 371 Date: Oct. 22, 2018

Related U.S. Application Data
(60) Provisional application No. 62/330,688, filed on May 2, 2016.

(51) Int. Cl. C07B 59/00; C07K 7/06

(52) U.S. Cl. CPC C07B 59/008; A61K 38/00; C07K 7/06

(57) ABSTRACT

This disclosure relates to isotopically modified amino acids and their pharmaceutical or nutritional uses in stabilizing pharmaceutical protein-based formulations, proteins with long life span, and preventing or treating disease such as Alzheimer's disease. Specifically, the disclosure provides protein drugs having increased stability comprising an L-aspartate (L-Asp) residue or an L-asparagine (L-Asn) residue, wherein the L-Asp residue or the L-Asn residue comprises a deuterium atom.

### INCORPORATION BY REFERENCE TO ANY PRIORITY APPLICATIONS

This application claims the benefit of U.S. Provisional Application No. 62/330,688, filed May 2, 2016, the disclosure of which is incorporated herein by reference in its entirety

### BACKGROUND

### Field

This disclosure relates to isotopically modified amino acids and their pharmaceutical or nutritional uses in stabilizing pharmaceutical protein-based formulations, proteins with long life span, and preventing or treating disease such as Alzheimer's disease.

### Description of the Related Art

One of the most prevalent spontaneous chemical reactions that lead to the degradation of proteins and peptides is the racemization of L-amino acids. The rate of racemization differs with each amino acid residue, with aspartic acid and asparagine residues being particularly susceptible. Racemization at aspartyl and asparaginyl residues could be responsible not only for the loss of function of protein pharmaceuticals during storage and use but for the loss of protein function in organisms, particularly for the long lived species in eye lens, and red blood cells. D-aspartate arising from L-aspartate and L-asparagine in the stable proteins of human tooth enamel accumulates with age and reaches about 8% of the total protein aspartate by 60 years of age, and D-aspartate in human eye lens crystallins increases to 10% of the total aspartate/asparagine residues over a 70-year lifespan. Protein D-aspartyl residues can be recognized by the protein repair L-isoaspartate-(D-aspartate) O-methyltransferase, an enzyme present in most organisms and cell types. However this reaction can lead to the formation of D-isoaspartyl residues and can thus enhance racemization.

D-aspartate can also accumulate in proteins that are prone to aggregate in several human pathologies. Proteins from brunescent cataracts have about twice the increase in the D-Asp/L-Asp ratio compared to non-cataractous lenses of comparable age. Additionally, the aspartyl and isoaspartyl residues in Alzheimer's disease brain neuritic plaque beta-amyloid have a D/L ratio of 11%. The abundance of racemized aspartyl and isoaspartyl residues is considerably less in vascular β-amyloid from these brains, suggesting that the vascular amyloid aggregates are “younger” than the neuritic amyloid plaques. These and other findings suggest that racemization of amino acid residues in long-lived proteins, particularly those arising from aspartyl and asparaginyl residues, might contribute to the loss of organ/tissue function with age and progression of diseases involving protein accumulation.

### SUMMARY

Some embodiments disclosed herein provide protein drugs having increased stability comprising an L-Asp residue or an L-Asn residue, wherein the L-Asp residue or the L-Asn residue comprises a deuterium atom. In some embodiments, the deuterium atom is located on the alpha carbon of the L-Asp residue or the L-Asn residue. In some embodiments, the deuterium atom is located on the side chain methylene group. In some embodiments, the L-Asp residue or the L-Asn residue comprises at least two deuterium atoms. In some embodiments, the L-Asp residue or the L-Asn residue comprises at least three deuterium atoms. In some embodiments, the L-Asp residue or the L-Asn residue comprises a deuterium atom at every hydrogen position. In some embodiments, the racemization rate of the L-Asp residue or the L-Asn residue is at least 50% lower than the racemization rate of an L-Asp residue or an L-Asn residue having no deuterium atoms. In some embodiments, the protein drug has a half life after administration into a human subject that is at least 20% greater than a corresponding protein drug having no deuterium atoms at the L-Asp residue or the L-Asn residue. In some embodiments, the protein drug has a stability that is at least 20% greater than a corresponding protein drug having no deuterium atoms at the L-Asp residue or the L-Asn residue.

Some embodiments disclosed herein provide methods of treating, ameliorating or preventing a disease in a human subject wherein the disease is caused by racemization of one or more Asp residues or one or more Asn residues in a protein, comprising administering to the human subject an effective amount of deuterated L-Asp or deuterated L-Asn. In some embodiments, at least 10% of the protein in the human subject is deuterated at the one or more Asp residues or the one or more Asn residues after the administration of deuterated L-Asp or deuterated L-Asn. In some embodiments, the racemization of the one or more Asp residues or the one or more Asn residues in the protein is decreased by at least 10% after the administration of deuterated L-Asp or deuterated L-Asn. In some embodiments, the disease is Alzheimer's disease. In some embodiments, the protein is amyloid-β. In some embodiments, the one or more Asp residues comprise Asp1, Asp7 and Asp23 of amyloid-β-42. In some embodiments, the one or more Asn residues comprise Asn27 of amyloid-β-42. In some embodiments, the deuterated L-Asp or deuterated L-Asn is administered daily.

Some embodiments disclosed herein provide products comprising a deuterated L-Asp or a deuterated L-Asn residue, wherein the amount of deuterium in the produce is above the naturally occurring level. In some embodiments, the racemization of the L-Asp residue or the L-Asn residue in the product is decreased by at least 10% when the product is in liquid form. In some embodiments, the product is a protein. In some embodiments, the product is Methyl L-α-aspartyl-L-phenylalaninate.

### BRIEF DESCRIPTION OF THE DRAWINGS

FIGS. 1A, 1B and 1C show (A) resonance forms of the succinimide product of asparagine deamidation showing the stabilization of the carbanion formed after removal of the proton on the alpha-carbon atom (modified from Radkiewicz et al. 1996), (B) structure of the deuterated Asn residue incorporated into the VYPNGA peptide, and (C) structure of d3-Fmoc-L-Asn(Tr)-OH, the deuterated Asn protected substrate for peptide synthesis.

FIG. 2 shows deamidation of deuterated asparagine in a synthetic peptide results in the generation of isoaspartyl- and normal aspartyl-containing peptides. Unaged VYPNGA containing protonated (dashed black line) or deuterated (dashed red line) coelute on a reverse-phase C18 HPLC column. Following incubation at 37° C. and pH 7.4 for 38.78 h, the amount of asparagine-containing peptide decreases as they are converted to Asp- and isoAsp-containing peptides. The L- and D-forms of these peptides are not separated under these conditions.

FIG. 3 shows deuteriums on the side chain methylene carbon and the alpha carbon of an asparaginyl residue have little effect on its rate of deamidation in a synthetic peptide. The peptide VYPNGA with either a protiated (upper panel) or deuterated (lower panel) asparaginyl residue was incubated at 37° C. in 0.1 M sodium phosphate pH 7.4 for various times. The relative quantity of asparagine-containing peptide remaining (open circles) was quantified by UV absorbance at 210 nm during separation of the peptides by HPLC using a reverse phase C18 column as shown in FIG. 2. There are 24 data points in each panel though some are concealed due to overlap, combined from two independent aging experiments. The line in each panel was fit to the data points using the first order reaction equation [A]=[A]₀e^{-kt} and least squares analysis. The half-lives of the peptides were calculated from the best-fit lines.

FIG. 4 shows the peptide containing deuterated asparagine accumulates racemized aspartate/isoaspartate/succinimide at a much slower rate than does the protiated peptide. VYPNGA containing either protiated or deuterated asparagine was incubated for various times at 37° C. and pH 7.4 and then acid hydrolyzed. The amount of D-aspartate and L-aspartate in the hydrolysates was quantified following derivatization with ortho-phthalaldehyde and N-acetyl-L-cysteine and separation of the resulting fluorescent diastereomers by reverse-phase HPLC, and graphed as the fraction of D-aspartate relative to the total aspartate (open circles). Each data set contains 24 measurements combined from two independent aging experiments; some of these data points are not visible due to overlap. The lines were obtained by computer simulation of the reactions involved in deamidation of asparaginyl residue starting with previously-published kinetic constants, and varying the racemization rate of the succinimide intermediate to give a best fit line as determined by least squares analysis.

### DETAILED DESCRIPTION

### Definitions

The term “neurodegenerative disease” as used herein, refers to any disease or abnormality of the of the nervous system caused by deterioration of neurons, which include death of neurons and functional loss of neurotransmitters. Non-limiting examples for a neurodegernative disease are Alzheimer's disease and Parkinson's disease. The term “food composition” as used herein, refers to any kind of composition which is eatable and/or drinkable without causing toxic symptoms in the subject eating or drinking the respective composition.

The term “late-onset Alzheimer's Disease” as used herein, refers to the onset of Alzheimer's Disease in elderly people, in particular in people being 65 years old and older.

The term “early-onset Alzheimer's Disease” as used herein, refers to the onset of Alzheimer's Disease in people younger than 65 years old.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Racemization in proteins and peptides at sites of L-asparaginyl and L-aspartyl residues can contribute to their spontaneous degradation, especially in the biological aging process. Amino acid racemization involves deprotonation of the alpha carbon and replacement of the proton in the opposite stereoconfiguration; this reaction is much faster for aspartate/asparagine than for other amino acids because these residues form a succinimide ring in which resonance stabilizes the carbanion resulting from proton loss. To determine if the replacement of the hydrogen atom on the alpha carbon with a deuterium atom might decrease the rate of racemization and thus stabilize polypeptides, a hexapeptide, VYPNGA, in which the three carbonbound protons in the asparaginyl residue were replaced with deuterium atoms was synthsized. Upon incubation of this peptide in pH 7.4 buffer at 37° C., it was found that the rate of deamidation via the succinimide intermediate was unchanged by the presence of the deuterium atoms. However, the accumulation of the d-aspartyl and d-isoaspartyl-forms resulting from racemization and hydrolysis of the succinimide was decreased more than five-fold in the deuterated peptideover a 20 day incubation at physiological temperature and pH. Additionally, it was found that the succinimide intermediate arising from the degradation of the deuterated asparaginyl peptide was slightly less likely to open to the isoaspartyl configuration than was the protonated succinimide. Therefore, the kinetic isotope effect resulting from the presence of deuteriums in asparagine residues may limit the accumulation of some of the degradation products that arise as peptides and proteins age. In some embodiments, the presence of at least one deuterium in asparagine or aspartate can help prevent or reduce the amount of the degradation products that arise as peptides and proteins age.

### Isotopically Modified Protein Drugs

Some embodiments disclosed herein provide protein drugs having increased stability. The protein described herein comprises an isotopically modified L-Asp residue or an L-Asn residue. In some embodiments, the L-Asp residue or the L-Asn residue comprises a deuterium atom. In some embodiments, the deuterium atom is located on the alpha carbon of the L-Asp residue or the L-Asn residue. In some embodiments, the protein is deuterated at one or more positions. In some embodiments, the protein is deuterated on the side chain methylene group.

In some embodiments, the protein described herein comprises an isotopically modified L-Ala residue, an isotopically modified L-Val residue, an isotopically modified L-Leu residue, an isotopically modified L-Ile residue, an isotopically modified L-Pro residue, an isotopically modified L-Phe residue, an isotopically modified L-Tyr residue, an isotopically modified L-Trp residue, an isotopically modified L-Ser residue, an isotopically modified L-Thr residue, an isotopically modified L-Cys residue, an isotopically modified L-Met residue, an isotopically modified L-Gln residue, an isotopically modified L-Lys residue, an isotopically modified L-Arg residue, an isotopically modified L-His residue, an isotopically modified L-Glu residue, or any combination thereof. In some embodiments, the protein described herein comprises an isotopically modified L-Ser residue.

In some embodiments, the racemization rate of the L-Asp residue or the L-Asn residue of the protein disclosed herein is at least 50% lower than the racemization rate of an L-Asp residue or an L-Asn residue having no deuterium atoms. In some embodiments, the racemization rate of the L-Asp residue or the L-Asn residue of the protein disclosed herein is at least 80% lower than the racemization rate of an L-Asp residue or an L-Asn residue having no deuterium atoms. In some embodiments, the racemization rate of the L-Asp residue or the L-Asn residue of the protein disclosed herein is at least 10%, at least 20%, at least 30%, at least 40%, at least 50%, at least 60%, at least 70%, at least 80%, at least 90%, at least 95%, at least 99%, lower than the racemization rate of an L-Asp residue or an L-Asn residue having no deuterium atoms.

In some embodiments, the protein drug has a half life after administration into a human subject that is at least 20% greater than a corresponding protein drug having no deuterium atoms at the L-Asp residue or the L-Asn residue. In some embodiments, the protein drug has a half life after administration into a human subject that is at least 50% greater than a corresponding protein drug having no deuterium atoms at the L-Asp residue or the L-Asn residue. In some embodiments, the protein drug has a half life after administration into a human subject that is at least 20%, at least 30%, at least 40%, at least 50%, at least 60%, at least 70%, at least 80%, at least 90%, at least 100%, at least 200%, at least 500%, at least 1,000%, at least 2,000%, greater than a corresponding protein drug having no deuterium atoms at the L-Asp residue or the L-Asn residue.

In some embodiments, the protein drug has a stability that is at least 20% greater than a corresponding protein drug having no deuterium atoms at the L-Asp residue or the L-Asn residue. In some embodiments, the protein drug has a stability that is at least 50% greater than a corresponding protein drug having no deuterium atoms at the L-Asp residue or the L-Asn residue. In some embodiments, the protein drug has a stability that is at least 20%, at least 30%, at least 40%, at least 50%, at least 60%, at least 70%, at least 80%, at least 90%, at least 100%, at least 200%, at least 500%, at least 1,000%, at least 2,000%, greater than a corresponding protein drug having no deuterium atoms at the L-Asp residue or the L-Asn residue.

In some embodiments, the protein drug can be a blood factor, a thrombolytic agent, a hormone, a haematopoietic growth factor, an interferon, an interleukin-based produce, a vaccine, an antibody, etc. In some embodiments, the protein drug can be abatacept, alefacept, erythropoietin, etanercept, denileukin diftitox, etc.

In some embodiments, the protein drug comprises an antibody. In some embodiments, the antibody is any form of antibody or fragment thereof that exhibits the desired biological activity, e.g., binding the specific target antigen. In some embodiments, the antibody can be monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab₂. In some embodiments, the antibody can be antibodies containing sequences of human origin, except for possible non-human complementarity-determining regions (CDR) regions, and does not imply that the full structure of an Ig molecule be present, only that the antibody has minimal immunogenic effect in a human.

In some embodiments, the antibody or antibody fragment can be abciximab, adalimumab, alemtuzumab, basiliximab, belimumab, bevacizumab, brentuximab vedotin, canakinumab, cetuximab, certolizumab pegol, daclizumab, daratumumab, denosumab, eculizumab, efalizumab, golimumab, ibritumomab tiuxetan, infliximab, ipilimumab, muromonab-CD3, natalizumab, nivolumab, ofatumumab, omalizumab, palivizumab, panitumumab, Pembrolizumab, ranibizumab, rituximab, tocilizumab (or atlizumab), tositumomab, trastuzumab, ustekinumab, or vedolizumab.

### Methods of Treating, Ameliorating or Preventing a Disease

Some embodiments disclosed herein provide methods of treating, ameliorating or preventing a disease in a human subject wherein the disease is caused by racemization of one or more Asp residues or one or more Asn residues in a protein. In some embodiments, the methods comprise administering to the human subject an effective amount of an isotopically modified compound. In some embodiments, the isotopically modified compound can be deuterated L-Asp or deuterated L-Asn.

In some embodiments, the disease is a neurodegenerative disease. In some embodiments, the disease is Alzheimer's disease. In some embodiments, the protein is an amyloid-β peptide. In some embodiments, the protein is an amyloid-β-42 peptide comprising the sequence Asp-Ala-Glu-Phe-Arg-His-Asp-Ser-Gly-Tyr-Glu-Val-His-His-Gln-Lys-Leu-Val-Phe-Phe-Ala-Glu-Asp-Val-Gly-Ser-Asn-Lys-Gly-Ala-Ile-Ile-Gly-Leu-Met-Val-Gly-Gly-Val-Val-Ile-Ala. In some embodiments, the one or more Asp residues comprise Asp1, Asp7 and Asp23 of amyloid-β-42. In some embodiments, the one or more Asn residues comprise Asn27 of amyloid-β-42.

In some embodiments, at least 10% of the protein in the human subject is deuterated at the one or more Asp residues or the one or more Asn residues after the administration of deuterated L-Asp or deuterated L-Asn. In some embodiments, at least 20% of the protein in the human subject is deuterated at the one or more Asp residues or the one or more Asn residues after the administration of deuterated L-Asp or deuterated L-Asn. In some embodiments, at least 10%, at least 20%, at least 30%, at least 40%, at least 50%, at least 60%, at least 70%, at least 80%, at least 90%, at least 95%, of the protein in the human subject is deuterated at the one or more Asp residues or the one or more Asn residues after the administration of deuterated L-Asp or deuterated L-Asn.

In some embodiments, the racemization of the one or more Asp residues or the one or more Asn residues in the protein is decreased by at least 10% after the administration of deuterated L-Asp or deuterated L-Asn. In some embodiments, the racemization of the one or more Asp residues or the one or more Asn residues in the protein is decreased by at least 20% after the administration of deuterated L-Asp or deuterated L-Asn. In some embodiments, the racemization of the one or more Asp residues or the one or more Asn residues in the protein is decreased by at least 10%, at least 20%, at least 30%, at least 40%, at least 50%, at least 60%, at least 70%, at least 80%, at least 90%, at least 95%, after the administration of deuterated L-Asp or deuterated L-Asn.

The deuterated L-Asp or deuterated L-Asn are preferably administered at a therapeutically effective dosage. While human dosage levels for the deuterated L-Asp or deuterated L-Asn described herein may vary, generally, a daily dose (or dosing 2, 3, or 4 times a day) of the deuterated L-Asp or deuterated L-Asn may be about the same as or 0.1, 0.15, 0.2, 0.3, 0.4, 0.5, 0.6, 0.7, 0.8, 0.9, 1.2, 1.4, 1.5, 1.75, 2, 3, 4, 5 or more times as much of the corresponding unmodified L-Asp or L-Asn the subject would typically ingest in their diet and produce via normal metabolism, displacing some or most or all of the unmodified L-Asp or L-Asn. It is preferred that the deuterated L-Asp or deuterated L-Asn comprise at least 10, 20, or 50% of the subject's daily L-Asp or L-Asn intake, and may comprise up to 100%, 200%, 500% or more of the subject's daily L-Asp or L-Asn intake. An objective of the therapy is to replace a significant amount of the L-Asp or L-Asn in the body with isotopically-modified L-Asp or L-Asn or metabolites thereof, such that at least 10%, 15%, 20%, 25%, 30%, 35%, 40%, 50% or more of L-Asp or L-Asn or L-Asp or L-Asn metabolites in the body is isotopically modified. Put another way, by repeatedly administering the isotopically-modified L-Asp or L-Asn to the patient, such that a significant percentage of the L-Asp or L-Asn or metabolites thereof in the body (or at a local site of interest) is isotopically-modified, the modified L-Asp or L-Asn will be resistant to racemization of one or more Asp residues or one or more Asn residues in a protein.

In some embodiments, the amount of the isotopically modified L-Asp or L-Asn administered to the subject is about 10%, 20%, 30%, 40%, 50%, 60%, 70%, 80%, 90%, 100%, 120%, 150%, 180%, or 200% of the amount of the unmodified L-Asp or L-Asn ingested by the subject. In some embodiments, the amount of the isotopically modified L-Asp or L-Asn administered to the subject is more than about 10%, 20%, 30%, 40%, 50%, 60%, 70%, 80%, 90%, 100%, 120%, 150%, 180%, or 200% of the amount of the unmodified L-Asp or L-Asn ingested by the subject. In some embodiments, the amount of the isotopically modified L-Asp or L-Asn administered to the subject is less than about 10%, 20%, 30%, 40%, 50%, 60%, 70%, 80%, 90%, 100%, 120%, 150%, 180%, or 200% of the amount of the unmodified L-Asp or L-Asn ingested by the subject. In some embodiments, the amount of the isotopically modified L-Asp or L-Asn administered to the subject is in the range of about 1-200%, 10%-200%, 10%-100%, or 20%-100% of the amount of the unmodified L-Asp or L-Asn ingested by the subject.

Administration of the deuterated L-Asp or deuterated L-Asn or compositions disclosed herein can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The deuterated L-Asp or deuterated L-Asn useful as described above can be formulated into pharmaceutical compositions, nutraceutical compositions, or food supplement. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected deuterated L-Asp or deuterated L-Asn useful as described above, some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

### Products

Some embodiments relate to produces comprising a deuterated L-Asp or a deuterated L-Asn residue, wherein the amount of deuterium in the produce is above the naturally occurring level. In some embodiments, the racemization of the L-Asp residue or the L-Asn residue in the product is decreased by at least 10% when the product is in liquid form. In some embodiments, the racemization of the L-Asp residue or the L-Asn residue in the product is decreased by at least 20% when the product is in liquid form. In some embodiments, the racemization of the L-Asp residue or the L-Asn residue in the product is decreased by at least 10%, at least 20%, at least 30%, at least 40%, at least 50%, at least 60%, at least 70%, at least 80%, at least 90%, at least 95%, when the product is in liquid form.

In some embodiments, the product described herein comprises an isotopically modified L-Ala residue, an isotopically modified L-Val residue, an isotopically modified L-Leu residue, an isotopically modified L-Ile residue, an isotopically modified L-Pro residue, an isotopically modified L-Phe residue, an isotopically modified L-Tyr residue, an isotopically modified L-Trp residue, an isotopically modified L-Ser residue, an isotopically modified L-Thr residue, an isotopically modified L-Cys residue, an isotopically modified L-Met residue, an isotopically modified L-Gln residue, an isotopically modified L-Lys residue, an isotopically modified L-Arg residue, an isotopically modified L-His residue, an isotopically modified L-Glu residue, or any combination thereof. In some embodiments, the protein described herein comprises an isotopically modified L-Ser residue.

In some embodiments, the products can be a protein. In some embodiments, the products can be a peptide or derivative thereof. In some embodiments, the products can be a bi-peptide or derivative thereof. In some embodiments, the products can be Methyl L-α-aspartyl-L-phenylalaninate.

### EXAMPLES

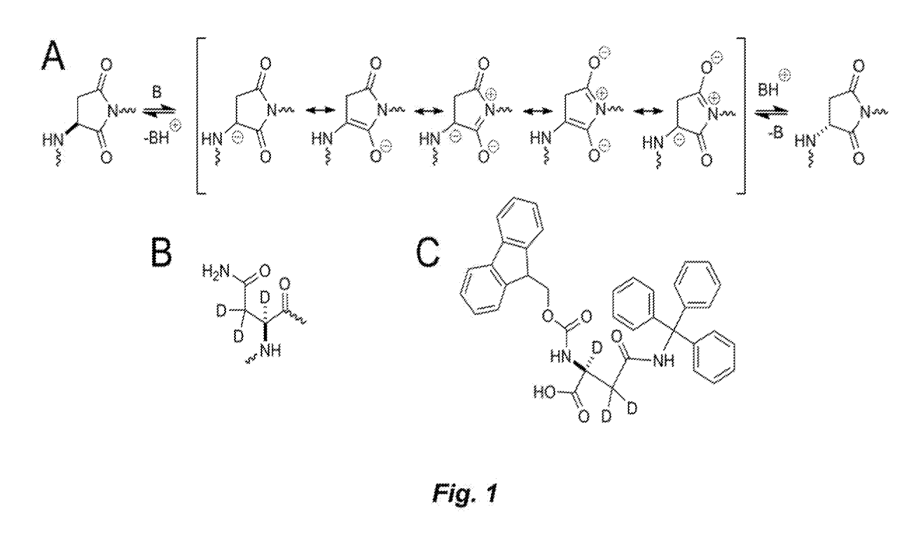

The mechanism by which aspartate and asparagine residues are particularly sensitive to racemization involves the reaction between the side chain carbonyl group with the peptide bond nitrogen of the following residue to produce a succinimide ring (Geiger and Clarke, (1987) J Biol Chem 262(2):785-794). Racemization occurs after the loss of a proton from the alpha carbon of this species to form a resonance-stabilized planar carbanion that can be reprotonated from either side to give a mixture of L- and D-succinimide (FIG. 1a). The change in the pKa of the proton on the alpha carbon has been supported by ab initio studies (Radkiewicz et al. (1996) J Am Chem Soc 118(38):9148-9155). The succinimide is also subject to hydrolysis at either of its two carbonyl carbons. Hydrolysis at the carbonyl that was formerly in the peptide's backbone occurs (at least in peptides) 75-80% of the time, resulting in an L- or D-isoaspartyl residue, in which the peptide backbone now goes through the former side-chain of the amino acid (Geiger and Clarke, supra; Stephenson and Clarke (1989) J Biol Chem 264(11):6164-6170; Manning et al. (1989) Pharm Res. 6(11):903-18). The rest of the time, hydrolysis occurs at the former side-chain carbonyl, generating either an L- or D-aspartyl residue. Recent work has also suggested that racemization can occur via a radical mechanism (Tambo et al. (2013) Biosci Biotechnol Biochem 77:416-418).

This technology relates to methods by which the potentially detrimental accumulation of racemized residues in proteins in the cell as well as in therapeutic peptide and protein preparations could be diminished. Classical kinetic isotope effects (KIE) occur when an atom is replaced by its heavy isotope, such as ²H or ¹³C. For non-enzymatic reactions, the deuterium KIE is typically between 2-9 (i.e. cleavage of a carbon-deuterium bond is 2-9 times slower than an equivalent carbon-proton bond) (Westheimer (1961) Chem. Rev. 61:265-273). It was proposed that deuteration at damage-vulnerable sites of biomolecules could slow down processes if hydrogen abstraction is the rate limiting step (Shchepinov (2007) Rejuvenation Research 10:47-59). Recently, it has been observed that deuteration of specific sites in polyunsaturated fatty acids (PUFA) inhibits lipid peroxidation, and supplementing Saccharomyces cerevisiae and cultured mammalian (H9C2) cells with deuterated PUFA protects these cells from oxidative stress (Hill et al. (2012) Free Radic Biol Med 53(4):893-906; Andreyev et al. (2015) Free Radic Biol Med 82:63-72). This protective effect is seen even when as little as 20% of the PUFAs have a single deuterium atom (Hill et al., supra). Thus, replacing the protons bound to the side chain methylene and alpha carbons of an L-asparaginyl residue in a polypeptide with deuterium atoms could potentially change the rate at which this residue undergoes deamidation via succinimide formation, the rate at which the L-succinimide racemizes to the D-succinimide form, and the tendency of the succinimide to open to the isoaspartyl form rather than the aspartyl form. A change in the rate of succinimide formation would result if these deuterium atoms change the nucleophilicity of the backbone nitrogen and/or the electrophilicity of the gamma carbonyl carbon involved the nucleophilic attack. Since racemization of the succinimide proceeds via the loss of the proton on the alpha carbon, replacing this hydrogen with a deuterium atom should decrease the concentration of the racemization-prone carbanion (Radkiewicz et al., supra). It is also possible that deuterium atoms on the beta carbon of the side chain could alter the resonance forms provided by the ring structure. Finally, deuteriums replacing the methylene group hydrogens in the succinimide ring could potentially alter the susceptibility of the two carbonyl groups by altering the resonance, or for steric reasons.

To determine how the presence of deuteriums might affect the deamidation, racemization, and isoaspartyl formation of an asparaginyl residue, peptides with the sequence L-Val-L-Tyr-L-Pro-L-Asn-Gly-L-Ala were generated, in which one peptide was made in the usual way and another made using a asparagine derivative where the hydrogens on the alpha carbon and the side chain methylene group were substituted with deuterium atoms (FIG. 1b). This amino acid sequence was chosen because the asparaginyl-, aspartyl-, and isoaspartyl-forms of this peptide can be separated and quantified by reverse-phase HPLC, and because the rates of succinimide formation and epimerization of the protonated peptide have been measured (Geiger and Clarke, supra; Stephenson and Clarke, supra). Although no effect of deuteration to the rates of succinimide formation was found, and only a small effect on the rates of succinimide hydrolysis was identified, a five-fold decreased rate of D-aspartate formation was detected, suggesting that deuteration can protect against racemization.

### General Methods

### Preparation of a Deuterated Asparagine Protected Derivative for Peptide Synthesis

d3-Fmoc-L-Asn(Tr)-OH (FIG. 1c) was prepared from d3-L-Asn(Trt)-OH.0.5H₂O and N-(9-fluorenylmethoxycarbonyloxy)succinimide as a white crystals (0.125 g, 77%) synthesized following the general procedure described in the literature (Albericio, F. (2000). Solid-Phase Synthesis: A Practical Guide (1 ed.). Boca Raton: CRC Press. p. 848. ISBN 0-8247-0359-6). M.p. 208-210° C. TLC: R_{f}0.35 (CHCl₃/MeOH/AcOH 94:5:1). Both the ¹H and ¹³C NMR spectra for d3-Fmoc-L-Asn(Trt)-OH were recorded in CDCl₃ on Bruker AV-400 instrument at room temperature. Chemical shifts are reported in ppm relative to TMS with the residual solvent peak used as an internal standard (references for DMSO-d₆: ¹H 2.50 ppm, ¹³C 39.43 ppm). ¹³C NMR spectrum was recorded with complete proton decoupling at 100.6 MHz.

¹H NMR (400 MHz, DMSO-d₆): δ=12.73 (br s, 1H), 8.64 (s, 1H), 7.90 (d, J=7.4 Hz, 2H), 7.73 (d, J=7.7 Hz, 2H), 7.63 (s, 1H), 7.47-7.11 (m, 19H), 4.44-4.11 (m, 3H). ¹³C NMR (100 MHz, DMSO-d₆): δ=173.1, 168.7, 155.7, 144.6 (×3), 143.7 (×2), 140.6 (×2), 128.5 (×6), 127.6 (×2), 127.3 (×6), 127.0 (×2), 126.3 (×3), 125.1 (×2), 120.0 (×2), 69.3, 65.6, 46.6.

### Peptide Synthesis

VYPNGA peptides were synthesized either with the deuterated Fmoc Asn derivative described above or with the standard Fmoc Asn derivative as trifluoroacetate salts by JPT Peptide Technologies, GmbH, Germany, with >95% purity, as determined by C18 reverse-phase HPLC with a linear gradient with detection at 220 nm. Mass spectrometric analyses using electrospray ionization confirmed the structure of both peptides with ions of 623.3 [M+H]⁺, 1244.6 [2M+H] for the deuterated peptide (calculated 622.68) and 620.3 [M+H]⁺, 1240.6 [2M+H]⁺ for the non-deuterated peptide (calculated 619.68).

### In Vitro Peptide Aging

Deuterated peptide VYPNGA and the protonated control peptide were dissolved in 0.1 M sodium phosphate, pH 7.44 at 23° C. These solutions were sterilized by passing them through 0.2 μm pore syringe filters (Fisher), and 50 μL aliquots containing 40 nmol of peptide were placed in 200 μL PCR tubes, which were incubated at 37° C. for various times up to 20.6 days. At this temperature, the pH of the phosphate buffer is very close to 7.40. At the end of the incubations, 4.5 μL of 1 M hydrochloric acid was added to each tube, lowering the pH to about 4 to minimize any additional deamidation of the asparaginyl residue. These aged samples were then stored at −20° C. prior to analysis by HPLC.

### Quantification of D-Aspartic Acid in Peptide Hydrolysates

First, a 10 μL aliquot (3.27 nmol) was removed from the aged sample and dried in a small glass test tube. These tubes were placed in a hydrolysis vial and 300 μL of 6 M hydrochloric acid was added to the vial but not directly into the sample tubes. The vial was capped with a Teflon stopcock, and then was flushed with nitrogen and sealed under vacuum using a PicoTag Work Station (Waters). This method allows only acid vapor to contact the peptide, minimizing contamination with exogenous aspartic acid. Hydrolysis proceeded at 110° C. for the relatively short time of 3 h, which is long enough to release most of the asparaginyl/aspartyl residues from the peptide, while minimizing acid catalyzed racemization (Brunauer and Clarke 1986). D-Aspartate and L-aspartate in the hydrolysates were then modified with o-phthalaldehyde and N-acetyl-L-cysteine to make fluorescent diasteriomers and quantitated using reverse phase HPLC with fluorometric analysis (Aswad 1984), as modified by Warmack et al. 2016.

### Determination of Racemization Rate Constants

To get an estimate of the racemization rate constants, the rate constants determined by Geiger and Clarke (1987) for a peptide of the same sequence was used but only with protonated asparagine using the following equations, where each k value has the units of min⁻¹:

L-Asn=Lasn-(Lasn*0.0003400)

L-imide=Limide+(Lasn*0.0003400)+(LAsp*0.0000099)+(Liso*0.0000099)+(Dimide*0.0002967)−(Limide*0.0002967)−(Limide*0.0039333)−(Limide*0.0010767)

L-isoAsp=Liso+(Limide*0.0039333)−(Liso*0.0000099)

L-Asp=Liso+(Limide*0.0039333)−(Liso*0.0000099)

D-imide=Dimide+(DAsp*0.0000125)+(Diso*0.0000125)+(Limide*0.0002967)−(Dimide*0.0002967)−(Dimide*0.0019333)−(Dimide*0.0006250)

D-isoAsp=Diso+(Dimide*0.0019333)−(Diso*0.0000125)

D-Asp=Dasp+(Dimide*0.0006250)−(Dasp*0.0000125)

At time zero, the l-asparaginyl-containing peptide is set to a quantity of 1.0, and all of the degradation products are considered to have a quantity of 0. The equations are then solved to give the quantities of the peptides present after 1 min, and these quantities are then used to solve the peptides for the next minute. Using Microsoft Excel, these calculations were repeated until the amount of each peptide present after 20.6 days was determined.

### Example 1

### Replacing the Protons of the Asparaginyl Residue in VYPNGA with Deuterium Atoms Does Not Affect Its Rate of Deamidation at pH 7.4 and 37° C.

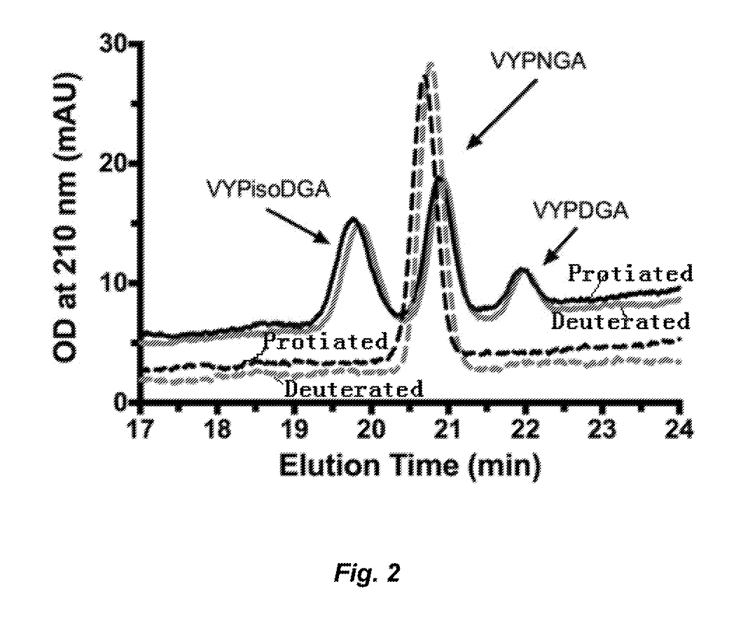

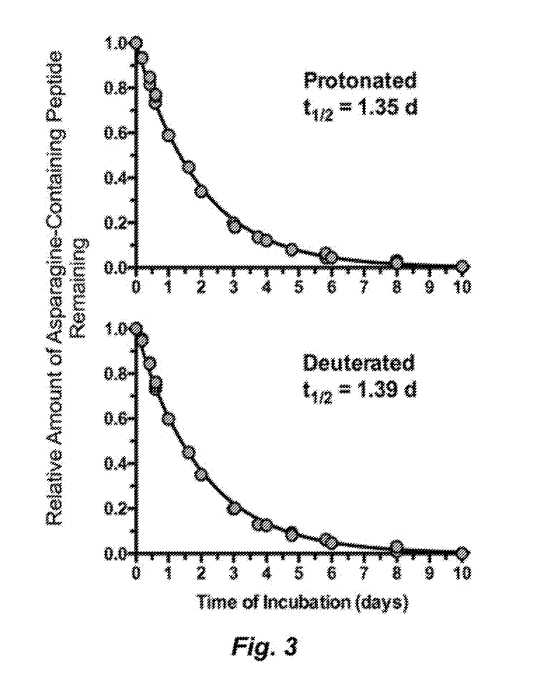

Deuterated and non-deuterated VYPNGA were aged in vitro in 0.1 M sodium phosphate at physiological pH and temperature. The rate of deamidation of the asparaginyl residue was determined by separating the peptide from its degradation products using reverse phase HPLC and measuring the UV absorbance at 210 nm using the approach of Geiger and Clarke, supra. It was observed that the deuterated peptide with intact asparagine elutes at the same position in the buffer gradient as the protonated peptide (FIG. 2). Upon incubation at pH 7.4 and 37° C., the primary products of spontaneous deamidation of the deuterated asparaginyl residue, the L-isoaspartyl- and L-aspartyl-containing peptides, also eluted at the same positions as the comparable protonated peptides (FIG. 2). The identity of the earlier eluting product of deamidation as the L-isoaspartyl-containing peptide was supported by its similar elution position to that determined by Geiger and Clarke, supra, for synthetic standards and the observation that it was approximately three-fold more abundant than the normal aspartyl-containing peptide eluting after the asparaginyl-containing peptide. This identification was confirmed for both the protonated and deuterated peptides by showing that only the early eluting peptide was a substrate for the recombinant human L-isoaspartyl methyltransferase (data not shown). Plotting the loss of the asparaginyl-containing peptide with time (FIG. 3) showed that the deamidation of the deuterated asparaginyl residue (t_{1/2}=1.39 days) proceeded at approximately the same rate as the protiated residue (t_{1/2}=1.35 days), and this rate was very similar to values that have been previously reported for the protiated peptide (t_{1/2}=1.42 days, Geiger and Clarke, supra; t_{1/2}=1.14 days, Stephenson and Clarke, supra). This suggests that the nucleophilicity of the glycyl residue's backbone nitrogen and the electrophilicity of the asparaginyl residue's side-chain carbonyl carbon are not affected by the deuterium atoms in such a way that the rate of the nucleophilic reaction that forms the succinimide is changed.

FIG. 2 shows that deamidation of deuterated asparagine in a synthetic peptide resulted in the generation of isoaspartyl- and normal aspartyl-containing peptides. Unaged VYPNGA (1.3 nmol) containing protiated (dashed black line) and deuterated (dashed gray line) asparagine residues were found to coelute on a reverse-phase C18 HPLC column (Alltech Econosphere, 5 μm beads, 250×4.6 mm) using a Hewlett Packard 1090 series II Liquid Chromatography system and monitoring UV absorption at 210 nm. Following incubation at 37° C. and pH 7.4 for 38.8 h, the amount of protiated (solid black line) and deuterated (solid gray line) asparagine-containing peptide (1.3 nmol) decreased as they were converted to aspartyl- and isoaspartyl-containing peptides. The l- and d-forms of these peptides were not separated under these conditions. These HPLC runs were done with a linear gradient from 100% buffer A (0.1% trifluoroacetic acid in water) to 86% buffer A/14% buffer B (0.1% trifluoroacetic acid, 0.9% water, 99% acetonitrile) in 28 min.

### Example 2

### The Presence of Deuterium Atoms Slightly Alters the Relative Rates of Hydrolysis at the Succinimide Intermediate's Two Carbonyl Carbons

Both isoaspartyl- and normal aspartyl-containing peptides arose from spontaneous deamidation of the deuterated asparaginyl residue upon incubation at pH 7.4 and 37° C. (FIG. 2), suggesting that the deamidation reaction proceeds via a succinimide intermediate as has been previously observed with protiated VYPNGA (Geiger and Clarke, supra). The succinimide ring contains two carbonyl carbons, both of which are sites of spontaneous hydrolysis; the half-life of this intermediate at physiological pH and temperature has previously been shown to be about 2.31 hours (Geiger and Clarke, supra). As seen in FIG. 2, hydrolysis of the succinimide ring favored creation of isoaspartate over normal aspartate in the product. When the quantities of isoaspartyl- and normal aspartyl-containing peptides were measured in the HPLC experiments of all samples that had been aged at 37° C. for at least 24 h, it was found that the average relative amount of isoaspartyl-containing peptide (isoAsp/(isoAsp+Asp)) was 0.793 (n=18; standard deviation 0.009) for the protiated deamidation products and 0.770 (n=17; standard deviation=0.012) for the deuterated deamidation products. Statistical analysis with the Student t-test showed that these values were significantly different (p=4.2×10⁻⁷ ). Thus, deamidation of the protiated and deuterated asparaginyl residues in our peptides both form isoaspartyl residues more frequently than normal aspartyl residues about 78.5% of the time as was previously reported for the protiated peptide (L-isoAsp/(L-isoAsp+L-Asp)=0.785; Geiger and Clarke 1987), but it was found that the presence of deuteriums rather than hydrogens on the succinimide intermediate improved hydrolysis at the former side chain carbonyl carbon, increasing the production of the normal aspartyl-containing peptide upon hydrolysis of the ring by about 2.3%.

In FIG. 3, deuteriums on the side chain methylene carbon and the alpha carbon of an asparaginyl residue had little effect on its rate of deamidation in a synthetic peptide. The peptide VYPNGA with either a protiated (upper panel) or deuterated (lower panel) asparaginyl residue was incubated at 37° C. in 0.1 M sodium phosphate pH 7.4 for various times. The relative quantity of asparagine-containing peptide remaining (open circles) was quantified by UV absorbance at 210 nm during separation of the peptides by HPLC using a reverse phase C18 column as shown in FIG. 2. There were 24 data points in each panel though some were concealed due to overlap, combined from two independent aging experiments. The line in each panel was fit to the data points using the first order reaction equation [A]=[A]0e−kt and least squares analysis. The half-lives of the peptides were calculated from the best-fit lines.

### Example 3

### The Presence of Deuteriums on the Asparaginyl Residue Significantly Decreases Its Rate of Racemization

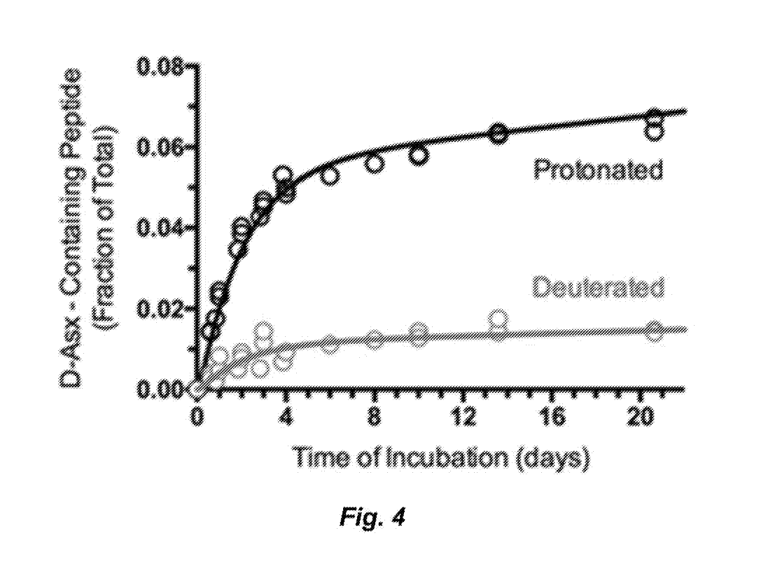

To investigate the effect of asparagine residue deuteration on racemization, the peptides were aged at 37° C. and pH 7.4 as described above, and the quantity of D-aspartate determined following acid hydrolysis. While breaking the peptide bonds to release free amino acids, acid hydrolysis also converts asparaginyl, succinimidyl, and isoaspartyl residues into free aspartic acid. As can be seen in FIG. 4, the peptide containing the deuterated asparaginyl residue is much more resistant to racemization than is the protiated peptide. The accumulation of D-Asx residues (D-Asp, D-isoAsp, and D-succinimide) was modeled using the rate constants as described in the “Materials and methods” section to fit the data points using the values obtained by Geiger and Clarke (1987) and maximizing the fit by varying the rate constant for succinimide racemization. Using least squares analysis to compare the modeled D-Asx accumulation to our experimentally measured values, it was determined a racemization rate constant of 3.4×10⁻⁴ min⁻¹, for the protiated peptide, a value about 1.14-fold higher than that previously measured (Geiger and Clarke, supra). When the data for the deuterated asparagine peptide was similarly analyzed, it was found a racemization rate constant of 7.4×10⁻⁵ min⁻¹, which is almost five-fold lower than that found for the protiated peptide.

The rate at which racemized residues accumulated in both the protiated and deuterated peptides is seen in FIG. 4 to be biphasic; the accumulation is rapid over the first four days, and then slower between six and twenty days. This occurs because asparaginyl residues in the protiated peptide VYPNGA form succinimides about 37-times more rapidly than does the aspartyl residue in VYPDGA (Stephenson and Clarke, supra). As shown in FIG. 2, the protiated and deuterated asparaginyl residues are 80% deamidated after three days and about 95% deamidated after 6 days, and thus the slower accumulation of racemized residues after six days reflects the slower succinimide formation of the now predominant aspartyl and isoaspartyl residues. Within the first day of aging under physiological conditions, well before half of the asparaginyl residues have deamidated, the rate of racemization of the protiated peptide is 3.65-times faster than that of the deuterated peptide. By 4 days of aging, when only about 14% of the peptides remain undeamidated, 5% of the Asn/Asp/isoAsp residues in the protiated peptide are racemized to the D-configuration, while only 1% of the deuterated Asn/Asp/isoAsp residues are racemized.

In FIG. 4, peptide containing deuterated asparagine accumulatd racemized aspartate/isoaspartate/succinimide at a much slower rate than the protiated peptide. VYPNGA containing either protiated or deuterated asparagine was incubated for various times at 37° C. and pH 7.4 and then acid hydrolyzed. The amount of d-aspartate and l-aspartate in the hydrolysates was quantified following derivatization with ortho-phthalaldehyde and N-acetyl-1-cysteine and separation of the resulting fluorescent diastereomers by reverse-phase HPLC, and graphed as the fraction of d-aspartate relative to the total aspartate (open circles). Each data set contained 24 measurements combined from two independent aging experiments; some of these data points were not visible due to overlap. The lines were obtained by computer simulation of the reactions involved in deamidation of asparaginyl residues starting with previously published kinetic constants (Geiger and Clarke 1987), and varying the racemization rate of the succinimide intermediate to give a best-fit line as determined by least squares analysis.

When the deuteron comes off the alpha carbon, there is a 50% chance that the proton that replaces it may come back to the same side of the alpha carbon, regenerating the L-succinimide, but now with only the two deuteriums on the methylene group remaining. Thus, there will now be a mixture of L-succinimide with three deuteriums and with two deuteriums, and these can hydrolyze to form L-isoaspartyl and L-aspartyl residues with three and two deuteriums, respectively. The deuterium is slowing down racemization because it is less likely to come off the alpha carbon than is a hydrogen. However, once the deuteron has come off and is replaced by a proton from the water, the rate of racemization will increase to the rate of racemization previously reported by Geiger and Clarke, supra. After the deuteron departs, the proton has a 50% chance of regenerating an L-succinimide, and this L-succinimide with an alpha hydrogen but with a deuterated methylene group may racemize as quickly as will a fully protonated L-succinimide. [0072] This l-succinimide with an alpha hydrogen but with a deuterated methylene group may racemize as quickly as a fully protonated l-succinimide. However, because the aspartyl/isoaspartyl/succinimidyl residues in the deuterated peptide are only 1.49% racemized to the D-configuration after 21 days of incubation, only 2×1.49%=2.98% of the succinimides generated during this incubation have lost the deuterium from the alpha carbon. The fact that over 97% of this deamidated peptide was still deuterated on the alpha carbon was observed in FIG. 4 by the slower rate of racemization between 9 and 22 days of incubation. The slope of this line for the deamidated deuterated peptides was about 1.70×10⁻⁴ day⁻¹, and the slope for the deamidated protonated peptides was 6.77×10⁻⁴ day⁻⁴. Thus, the rate of racemization of l-aspartyl/lisoaspartyl residues was about fourfold slower when these residues arise from deuterated l-asparaginyl residues.

### Conclusions

It was shown here that the replacement of hydrogen atoms with deuterium atoms can markedly reduce the degree of spontaneous racemization in a model asparagine peptide. However, such replacement does not decrease the rate of deamidation and the consequent rate of formation of isomerized residues. However, isomerization damage can be readily reversed by the action of the protein repair methyltranferase in converting L-isoaspartyl residues to normal L-aspartyl residues. But, the repair of D-aspartyl residues is much less efficient, and leads to the generation of D-isoaspartyl residues (Lowenson and Clarke (1992) J Biol Chem 267(9):5985-95, Young et al. (2005) J Biol Chem 280(28):26094-8) so that preventing D-aspartyl formation in the first place by deuteration may be important in stabilizing protein and peptide pharmaceuticals and cellular proteins. The finding reported here suggests a potential intervention strategy to minimize the damage.

All publications, patents, and patent applications mentioned in this specification are herein incorporated by reference for all purposes to the same extent as if each individual publication, patent, or patent application was specifically and individually indicated to be incorporated by reference.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A protein drug having increased stability comprising an L-Asp residue or an L-Asn residue, wherein the L-Asp residue or the L-Asn residue comprises a deuterium atom.

2. The protein drug of claim 1, wherein the deuterium atom is located on the alpha carbon of the L-Asp residue or the L-Asn residue.

3. The protein drug of claim 1 or 2, wherein the deuterium atom is located on the side chain methylene group.

4. The protein drug of any one of claims 1 to 3, wherein the L-Asp residue or the L-Asn residue comprises at least two deuterium atoms.

5. The protein drug of any one of claims 1 to 4, wherein the L-Asp residue or the L-Asn residue comprises at least three deuterium atoms.

6. The protein drug of any one of claims 1 to 5, wherein the L-Asp residue or the L-Asn residue comprises a deuterium atom at every hydrogen position.

7. The protein drug of any one of claims 1 to 6, wherein the racemization rate of the L-Asp residue or the L-Asn residue is at least 50% lower than the racemization rate of an L-Asp residue or an L-Asn residue having no deuterium atoms.

8. The protein drug of any one of claims 1 to 7, wherein the protein drug has a half life after administration into a human subject that is at least 20% greater than a corresponding protein drug having no deuterium atoms at the L-Asp residue or the L-Asn residue.

9. The protein drug of any one of claims 1 to 8, wherein the protein drug has a stability that is at least 20% greater than a corresponding protein drug having no deuterium atoms at the L-Asp residue or the L-Asn residue.

10. A method of treating, ameliorating or preventing a disease in a human subject wherein the disease is caused by racemization of one or more Asp residues or one or more Asn residues in a protein, comprising administering to the human subject an effective amount of deuterated L-Asp or deuterated L-Asn.

11. The method of claim 10, wherein at least 10% of the protein in the human subject is deuterated at the one or more Asp residues or the one or more Asn residues after the administration of deuterated L-Asp or deuterated L-Asn.

12. The method of claim 10 or 11, wherein the racemization of the one or more Asp residues or the one or more Asn residues in the protein is decreased by at least 10% after the administration of deuterated L-Asp or deuterated L-Asn.

13. The method of any one of claims 10 to 12, wherein the disease is Alzheimer's disease.

14. The method of any one of claims 10 to 13, wherein the protein is amyloid-β.

15. The method of claim 14, wherein the one or more Asp residues comprise Asp1, Asp7 and Asp23 of amyloid-β-42.

16. The method of claim 14, wherein the one or more Asn residues comprise Asn27 of amyloid-β-42.

17. The method of any one of claims 10 to 16, wherein the deuterated L-Asp or deuterated L-Asn is administered daily.

18. A product comprising a deuterated L-Asp or a deuterated L-Asn residue, wherein the amount of deuterium in the produce is above the naturally occurring level.

19. The product of claim 18, wherein the racemization of the L-Asp residue or the L-Asn residue in the product is decreased by at least 10% when the product is in liquid form.

20. The product of claim 18 or 19, wherein the product is a protein.

21. The product of any one of claims 18 to 20, wherein the product is Methyl L-α-aspartyl-L-phenylalaninate.
